(19) United States
(12) Patent Application Publication
(10) Pub. No.: US 2007/0025942 A1
(43) Pub. Date: Feb. 1, 2007

(54) Clear gel antiperspirant or deodorant compositions and related methods

(76) Inventors: Vijay Kumar Joshi, Livingston, NJ (US); Philip Franco, Ocean Grove, NJ (US); Debra Kutylowski, Forked River, NJ (US)

Correspondence Address: Julie Blackburn;Revlon Consumer Products Corporation, 237 Park Avenue, New York, NY, 10017, US

(21) Appl. No.: 11/189,618

(22) Filed: Jul. 26, 2005

(51) Int. Cl. A61K 8/28; A61K 8/26

(52) U.S. Cl. 424/65; 424/66; 424/68

(57) ABSTRACT

A clear gel emulsion antiperspirant or deodorant composition comprising at least one antiperspirant or deodorant active, a water phase, and an oil phase containing at least one high refractive index oil, wherein the composition is prepared from an oil phase and a water phase that, prior to emulsification, are both optically clear; a clear gel emulsion antiperspirant or deodorant composition containing at least one polymeric alpha olefin in the oil phase; and a method for improving the clarity of a clear gel emulsion antiperspirant or deodorant composition.

### TECHNICAL FIELD

The invention is in the field of antiperspirant or deodorant compositions, particularly those that are optically clear, and methods for formulating gel antiperspirant or deodorant compositions with improved clarity.

### BACKGROUND OF THE INVENTION

Clear gels are a popular delivery vehicle for antiperspirants and deodorants. One simple reason for this is that consumers perceive that when such formulas are clear they will not leave a residue on underarms or clothing. There are a wide variety of commercially available antiperspirants and deodorants in the clear gel form. It is a very popular form with consumers for a variety of other reasons. For example, clear gels are aqueous based so they provide a cooling, aesthetically pleasing feel when applied to skin. They are inexpensive to manufacture and because the gel is in a liquid or semi-solid form it eliminates many of the difficult processing issues that may occur in stick manufacture.

One common problem with clear gels, however, is that they may tend to become cloudy or turbid with time. Sometimes the gel yellows. In some cases it is believed that, with respect to water and oil emulsion clear gels, that the decrease in clarity with time may be due to the fact that during manufacture, prior to emulsification, the oil phase and/or the water phase used to prepare the gel are not initially clear. For example, in most emulsion clear gel formulas, the oil phase and the water phase are separately prepared. The oil phase is often cloudy because of the oils or waxes that are used in the formulation, while the water phase is typically clear. When the two phases are combined, the refractive index of the phases is adjusted to provide a final composition that is substantially clear. As the gel ages it is believed that the emulsion may deteriorate slightly, causing the separate phases to revert to their normal character. That, in turn, causes the gel to become cloudy or turbid.

As consumers associate clarity with desirable attributes they will not purchase antiperspirant or deodorant products that have become turbid or yellowed, even though the products may still be just as efficacious. Rather, the products are sent back to the manufacturer as returns, which is expensive and time consuming for both the retailer and the cosmetics manufacturer.

Accordingly, cosmetics formulators are always looking for ways to improve the clarity and aesthetics of clear gel formulas without sacrificing properties such as spreadability, feel, aesthetics, and the like.

It has been discovered that the clarity of emulsion clear gel antiperspirants or deodorants can be significantly improved when both the oil and water phases of the gels are clear prior to emulsification.

It has also been discovered that formulating clear gel antiperspirants or deodorants with high refractive index oils in the oil phase substantially improves clarity and provides products that have excellent aesthetic properties.

It is an object of the invention to provide a method for improving clarity of clear gel antiperspirants or deodorants comprising formulating oil and water phases that are essentially clear before emulsification, and which result in a clear gel after emulsification.

It is a further object of the invention to provide a method for improving clarity of clear gel antiperspirants or deodorants by including in such compositions at least one high refractive index oil, such as a polymeric alpha olefin, in the oil phase.

It is a further object of the invention to provide an aqueous based clear gel antiperspirant or deodorant composition comprising at least one high refractive index oil, such as a polymeric alpha olefin, in the oil phase of the emulsion.

### SUMMARY OF THE INVENTION

The invention is directed to a clear gel emulsion antiperspirant or deodorant composition comprising at least one antiperspirant or deodorant active, a water phase, and an oil phase containing at least one high refractive index oil, wherein the composition is prepared from an oil phase and a water phase that, prior to emulsification, are both optically clear.

The invention is further directed to a clear gel emulsion antiperspirant or deodorant composition comprising at least one antiperspirant or deodorant active, a water phase, and an oil phase containing at least one polymeric alpha olefin.

The invention is further directed to a method improving the clarity of a clear gel emulsion antiperspirant or deodorant composition comprising separately preparing an optically clear water phase and an optically clear oil phase containing at least one high refractive index oil, and emulsifying the phases to provide an optically clear final composition.

The invention is further directed to a method for improving the clarity of a clear gel emulsion antiperspirant or deodorant composition comprising separately preparing an optically clear water phase and an optically clear oil phase containing at least one polymeric alpha olefin, and emulsifying the phases to provide an optically clear composition.

### DETAILED DESCRIPTION

### I. Definitions

A. All percentages used herein are percentages by weight unless otherwise indicated.

B. The term “antiperspirant” means a product that treats perspiration by inhibiting it, usually by use of astringent antiperspirant salts.

C. The term “astringent antiperspirant salt” or “antiperspirant salt” means a salt that has astringent, or pore contracting, activity on sweat glands and pores and interferes with their ability to release perspiration. The most commonly used antiperspirant salts are typically inorganic or organic salts of aluminum, zirconium, and zinc, and mixtures thereof.

D. The term “clear” when used herein means that the composition is optically clear, or clear when viewed with the naked eye.

E. The term “deodorant” means a composition that ameliorates body malodor by inhibiting the cause of the malodor, which is the bacteria that resides on the skin.

F. The term “gel” means a composition that has the consistency of a liquid, or a semi-solid that flows or migrates upon application of pressure.

G. The term “high refractive index” means that the oil exhibits a refractive index of about 1.0 to 1.5, and is most preferably optically clear. The term “optically clear” means that when viewed with the naked eye the oil is clear or substantially free from clouds, mist, or haze.

H. “Nephelometric turbidity units” means the degree of turbidity in the composition when measured by ASTM Test Method D6855-03, Standard Test Method for Determination of Turbidity Below 5 NTU in Static Mode.

I. The term “non-volatile” means that the ingredient has a vapor pressure of less than about 2 millimeters of mercury at 20° C.

J. “Oil” means an ingredient that is pourable at room temperature.

K. “Refractive Index” means a value obtained by measuring the angle of incidence and the angle of refraction and applying the formula n=sin(θᵢ)/sin(θᵣ), where n is the index of refraction. The refractive index of the compositions of the invention are preferably measured using an Abbe refractometer when the temperature of the sample is maintained at room temperature (25° C.).

L. The term “volatile” means that the ingredient has a vapor pressure of greater than about 2 millimeters of mercury at 20° C.

II. The Composition

A. Water

The composition of the invention may be in the solution or emulsion form. In each case, the composition preferably comprises from about 0.1-99%, preferably from about 5-95%, more preferably from about 10-90% by weight of the total composition of water.

B. Astringent Antiperspirant Salt

If the composition of the invention is an antiperspirant, the composition comprises one or more astringent antiperspirant salts. Suggested ranges are from about 0.1-85%, preferably from about 1-75%, more preferably from about 5-70% by weight of the total composition.

Suitable astringent antiperspirant salts are compounds or compositions having antiperspirant activity, preferably metallic salts such as the inorganic and organic salts of aluminum, zirconium, and zinc, and mixtures thereof. Particularly preferred are the aluminum and zirconium salts such as aluminum halides, aluminum hydroxy halides, zirconyl oxide halides, zirconyl hydroxy halides, and mixtures thereof. Aluminum salts include those of the formula:
Al₂(OH)ₐCl_{b}.xH2O
wherein a is from about 2 to 5; a+b=6; x is from about 1 to about 6; and wherein a, b, and x may have non-integer values. Zirconium salts include those of the formula:
ZrO(OH)₂₋ₐClₐ.xH₂O
wherein a is from about 1.5 to about 1.87; x is from about 1 to about 7; and wherein a and n may have non-integer values.

Examples of aluminum and zirconium salts include aluminum chloride, aluminum chlorohydrate, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum zirconium octachlorohdrate, aluminum zirconium octachloroydrex GLY, aluminum zirconium pentachlorohydrate, aluminum zirconium pentachlorohydrex GLY, aluminum zirconium tetrachlorohydrate, aluminum zirconium tetrachlorohydrex GLY, aluminum zirconium trichlorohydrate, aluminum zirconium trichlorohydrex GLY, and mixtures thereof.

More specific types of zirconium salts are those complexes also containing aluminum and glycine, in particular, aluminum zirconium tetrachlorohydrex GLY.

Preferred for use in the composition are astringent antiperspirant salts that are halides or halohydrates of aluminum such as aluminum chloride or aluminum chlorohydrate.

The antiperspirant salts used in the composition of the invention are preferably solubilized in the water phase. While preferably the antiperspirant salts are completely dissolved in the water, in some cases small amounts of salts may not be dissolved, i.e. may remain in the crystalline or suspensoid form.

C. Deodorant Actives

If the composition of the invention is in the form of a deodorant, it will contain one or more deodorant actives, which are ingredients that effectively inhibit the bacterial growth on the skin. Suitable ranges of deodorant actives are from about 0.1-30%, preferably from about 0.5-28%, more preferably from about 1-25% by weight of the total composition.

Preferably, the deodorant actives are soluble or dispersible in the water phase of the composition. Examples of suitable deodorant actives include fragrances, ammonium phenolsulfonate, benzalkonium chloride, benzethonium chloride, bromochlorophene, cetylpyridinium chloride, chlorophyllin-copper complex, chlorothymol, chloroxylenol, cloflucarban, dequalinium chloride, dichlorophene, dichloro-m-xylenol, disodium dihydroxyethyl sulfosuccinylundecylenate, domiphen bromide, hexachlorophene, lauryl pyridinium chloride, methylbenzethonium chloride, phenol, sodium bicarbonate, sodium phenolsulfonate (triclocarbone), triclocarban, triclosan, zinc phenolsulfonate, zinc ricinoleate, and mixtures thereof. In the case where the composition of the invention is a deodorant, the preferred deodorant active is triclosan, fragrance, or mixtures thereof.

It is also possible for the composition to have both antiperspirant and deodorant properties. In this case, the composition will contain both antiperspirant salts and deodorant active ingredients.

D. The High Refractive Index Oil

The compositions of the invention comprises from about 0.01-99%, preferably from about 0.5-95%, more preferably from about 1-85% by weight of the total composition of one or more high refractive index oils. Examples of such oils include polymeric alpha olefins, esters of C₁₋₆ alcohols, or copolymers of alpha olefins and ethylenically unsaturated monomers such as vinyl pyrrolidone and the like. In the case where copolymers of alpha olefins and ethylenically unsaturated monomers are used, such ingredients may not be in a pourable liquid form. For example, one type of polymer suitable for use as the high refractive index material is tricontanyl PVP, which is in a solid flake form. However, when it is dissolved in silicone oil or other esters or hydrocarbons (whether clear or not) it forms a clear, pourable liquid that is suitable for use in the composition as the high refractive index oil. In the case where the high refractive index oil is obtained by combining copolymers of alpha olefins and ethylenically unsaturated monomers that are initially in the solid or flake form, the composition must contain an oil in an amount sufficient to solubilize the polymer to form a high refractive index oil having the desired optical clarity.

1. Polymeric Alpha Olefins

Examples of suitable high refractive index oils include polymeric alpha olefins, such as those formed from hydrocarbons having from 2 to 20 carbon atoms and with olefinic unsaturation between the first two carbon atoms of the hydrocarbon chain. Examples of such monomers include butene (C4), hexene (C6), octene (C8), decene (C 10), dodecene (C 12). tetradecene (C 14) and the like. The number of repeating monomers in the polymer may range from 2 to about 1000, including all whole numbers in between; more preferably from about 2 to about 100. Most preferred is where the polymeric alpha olefin is made from monomers such as butene or decene where the number of repeating monomer units is 2, 4, 6, 8, 10, 12, or 14. If desired one or more of the monomer units may be hydrogenated, e.g. because one or more monomers in the final polymer have been hydrogenated those monomers do not contain olefinic unsaturation.

Preferred is where the composition contains one or more polymeric alpha olefins having from two to four repeating alpha olefin monomer units, more preferably, where the monomers are butene or decene. More preferred are polymeric alpha olefins such as polydecene-2, polydecene-4, or mixtures thereof, having two and four repeating decene units respectively. Such polymers may be purchased from ExxonMobil under the trade name PureSyn™ PAO (2-100) with the designation 2 to 100 meaning the number of repeating alpha olefin monomers. With respect to such polydecenes, they generally have a specific gravity ranging from about 0.7 to 0.9 at 15.6° C.; a Brookfield viscosity ranging from about 5 to 3,000 at 25° C.; a refractive index ranging from about 1.4 to about 1.5 at 25° C. Most preferred is where the one or more polymeric alpha olefins present range from about 1-4% by weight of the total composition.

2. Esters

Also suitable as the high refractive index oil are various esters obtained by the reaction of lower alkanols and lower chain carboxylic acids. Examples of such esters include those obtained by reaction C₁₋₆ straight or branched chain alkanols with C₁₋₂₅ straight or branched chain carboxylic acids. Further examples of such esters are those obtained by the reaction of C₂, C₃, or C₄ alkanols such as ethanol, isopropyl alcohol, or propanol, with a C₁₋₂₀ straight or branched chain, saturated or unsaturated mono- or di- or tricarboxylic acids. Further examples of such suitable esters include those obtained by the reaction of a C₂₄ alkanol with dicarboxylic acids such as succinic acid, glutaric acid, adipic acid, sebacic acid, and so on. Particularly preferred are diisopropyl adipate (refractive index about 1.42), diisopropyl sebacate (refractive index about 1.43), dibutyl succinate (refractive index about 1.42), or mixtures thereof.

3. Copolymers of Alpha Olefins and Ethylenically Unsaturated Monomers

Also suitable are certain copolymers of alpha olefins and various ethylenically unsaturated monomers. Such polymers may be in the clear liquid form as is, or may form clear liquid mixtures when dissolved in another silicone, ester, or hydrocarbon oil. Examples of such copolymers include copolymers of C₁₋₂₀ alpha olefins and ethylenically unsaturated monomers such as vinyl pyrrolidone, ethylene, propylene, acrylic acid esters, and the like. Particularly preferred are copolymers of C₂₋₁₀₀ alpha olefins and PVP (polyvinylpyrrolidone) sold by International Specialty Products (ISP) under the trade name Ganex. Examples of such polymers include PVP/eicosene copolymer, Tricontanyl PVP, and the like.

E. Water Soluble Solvents

The composition may also contain one or more water soluble solvents. If present, such solvents may range from about 0.01-60%, preferably from about 0.05-55%, more preferably from about 0.1-50% by weight of the total composition. Examples of suitable solvents include mono-, di-, or polyhydric alcohols, di C₁₋₁₀ alkylene glycols, and the like.

1. Mono-, Di-, or Polyhydric Alcohols

Suitable monohydric alcohols include C₂₋₁₀ straight or branched chain, saturated or unsaturated alkanols. Such monohydric alcohols may be primary, secondary, or tertiary, with that designation referring to the number of carbon atoms bonded to the carbon atom bearing the hydroxyl substituent. For example, ethanol, having the following formula: 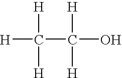
is a primary monohydric alcohol because it has one hydroxyl group and one carbon atom bonded to the carbon atom which bears the hydroxyl group. On the other hand, isopropanol, having the following formula: 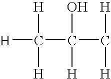
is an example of a monohydric secondary alcohol because it has one hydroxyl group and two carbon atoms bonded to the carbon atom bearing the hydroxyl group.

Examples of monohydric alcohols suitable for use in the composition of the invention include ethanol, isopropanol, butanol, pentanol, hexanol, and so on.

Suitable dihydric alcohols, or alcohols having two hydroxyl groups in the molecule, include C₂₋₁₀ straight or branched chain alkanols. As with the suitable monohydric alcohols, they may be primary, secondary, or tertiary. Examples of suitable dihydric alcohols include ethylene glycol, propylene glycol, butylene glycol, pentylene glycol, hexylene glycol, or mixtures thereof.

Suitable polyhydric alcohols are those having three or more hydroxyl groups per molecule. One example of such a polyhydric alcohol is glycerin.

2. Polymeric Alkylene Glycols

Also suitable are dialkylene glycols, or glycols having more than one repeating dihydric or polyhydric alcohol. Examples of such ingredients include PEG₂₋₂₀₀ (polyethylene glycol having 2 to 200 repeating ethylene glycol units); PPG2-200 (polypropylene glycol having from 2 to 200 repeating propylene glycol units); and so on.

The preferred compositions of the invention contain one or more of a polyhydric alcohol, a dihydric alcohol, and a polymeric alkylene glycol. More particularly, the preferred compositions of the invention contain a polyhydric alcohol which is glycerin, a dihydric alcohol which is propylene glycol, and a polymeric alkylene glycol which is dipropylene glycol.

F. Other Oils

Preferably, the compositions of the invention are in the form of emulsions, either water in oil or oil in water. In addition to the percentage ranges of water and the at least one high refractive index oil, the composition may contain from about 0.1-99%, preferably from about 0.5-95%, more preferably from about 1-90% by weight of the total composition of other oils besides the one or more high refractive index oil. A variety of oils are suitable, includes hydrocarbons, esters, silicone oils, and the like.

1. Silicone Oils

Suitable silicone oils include volatile and non-volatile linear or cyclic silicones.

(a). Volatile Silicone Oils

Cyclic silicones (or cyclomethicones) are of the general formula: 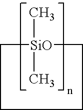
where n=3-6.

Also suitable are linear volatile silicones in accordance with the invention, which have the general formula:
(CH₃)₃Si—O—[Si(CH₃)₂—O]ₙ—Si(CH₃)₃
where n=0-6, preferably 0-5.

Linear and cyclic volatile silicones are available from various commercial sources including Dow Coming Corporation and General Electric. The Dow Coming volatile silicones are sold under the tradenames Dow Coming 244, 245, 344, and 200 fluids. These fluids comprise octamethylcyclotetrasiloxane, decamethylcyclopentasiloxane, dodecamethylcyclohexasiloxane, hexamethyldisiloxane, octamethyltrisiloxane, decamethyltetrasiloxane, dodecamethylpentasiloxane, and mixtures thereof.

(b). Non-Volatile Silicone Oils

Also suitable are various non-volatile silicones. Such silicones preferably have the general formula: 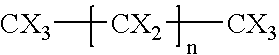
wherein n is 5 or greater, preferably from about 5 to 1,000,000; and each X is independently H, phenyl, trimethylsiloxy, fluoro, or C₁₋₁₀ alkoxy, with methoxy, ethoxy, propoxy, and butoxy being examples. Further, more specific examples of such silicone oils include those referred to as dimethicone, phenyl trimethicone, phenyl dimethicone, diphenyl dimethicone, and the like.

2. Paraffinic Hydrocarbons

Also suitable as other oils are various volatile or non-volatile paraffinic hydrocarbons.

(a). Volatile Paraffinic Hydrocarbons

Suitable volatile paraffinic hydrocarbons include those having straight or branched chains having about 5 to 18 carbon atoms, more preferably about 8-18 carbon atoms. Examples include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of about 70-225, preferably about 160 to 190 and a boiling point range of about 30 to 320° C., preferably 60-260° C., and a viscosity of less than about 10 centipoise at 25° C. Such paraffinic hydrocarbons are available from EXXON under the ISOPARS trademark, and from the Permethyl Corporation. Suitable C₁₂ isoparaffins (isododecane) are manufactured by Permethyl Corporation under the tradename Permethyl 99A. Various C₁₆ isoparaffins commercially available, such as isohexadecane (having the tradename Permethyl R), are also suitable.

(b). Non-Volatile Paraffinic Hydrocarbons

It may be desired to include one or more non-volatile paraffinic hydrocarbons as the other oil in the composition. Examples of such hydrocarbons include straight or branched chain hydrocarbons having from 18 to 40 carbon atoms such as heneicosane, docosane, n-octadecane, nonadecane, eicosane, tetracosane, pentacosane, hexacosane, heptacosane, octacosane, dotriacontane, tritriacontane, hexatriacontane, hydrogenated polyisobutene, mineral oil, pentahydrosqualene, squalene, squalane, and so on.

3. Esters

Also suitable as the other oil are various esters that are not optically clear at room temperature (as opposed to the esters described herein as high refractive index oils). Such esters may be mono-, di-, or triesters.

(a). Monoesters

Suitable monoesters are generally formed by the reaction of a monocarboxylic acid and an aliphatic alcohol that may be substituted with one or more substituents such as hydroxyl, alkyl, or alkoxy groups. Such esters preferably have the general formula R₁-COOR₂ wherein R₁ and R₂ are each independently a C₁₋₄₅ straight or branched chain, saturated or unsaturated alkyl, alkoxy, C₁₋₃₀ alkoxy alkyl, and the like, any of which such mentioned substituents may be substituted with hydroxyl, C₁₋₃₀ alkyl, or C₁₋₃₀ alkoxy groups. Examples of such monoesters include monoesters of fatty acids having from 6 to 30 carbon atoms, such as stearic acid, malic acid, oleic acid, linoleic acid, behenic acid, palmitic acid, myristic acid, and so on. Further examples of monoesters include isostearyl malate, isopropyl palmitate, stearyl stearate, isopropyl malate, hexyl laurate, cetyl isononanoate, butyl oleate, cetyl palmitate, hexadecyl octanoate, and so on.

(b). Diesters

Suitable diesters that may be used in the compositions of the invention are the reaction product of a dicarboxylic acid and an aliphatic or aromatic alcohol, or alternatively, the reaction product of a monocarboxylic acid and an aliphatic or aromatic alcohol having at least two hydroxyl groups. The dicarboxylic acid or the alcohol may contain from 2 to 45 carbon atoms, and may be in the straight or branched chain, saturated or unsaturated form. In the case where the ester is formed from a dicarboxylic acid, it may be subsituted with one or more hydroxyl groups. The aliphatic or aromatic alcohol may also contain from 2 to 30 carbon atoms, and may be in the straight or branched chain, saturated, or unsaturated form. The aliphatic or aromatic alcohol may also be substituted with one or more substituents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, di-C₁₂₋₁₃ alkyl malate, dicetearyl dimer dilinoleate, diisostearyl palmitate, disostearyl fumarate, and so on.

(iii). Triesters

Suitable triesters that may be used in the compositions include those that are the reaction product of a tricarboxylic acid and an aliphatic or aromatic alcohol, or the reaction product of a mono- or dicarboxylic acid and an aliphatic alcohol having two, three, or more substituted hydroxyl groups. As with the mono- and diesters mentioned above, either the acid or the alcohol or both may contain from about 2 to 30 carbon atoms, and may be saturated or unsaturated, straight or branched chain, and may be substituted with one or more hydroxyl groups. Preferably, one or more of the acid or alcohol is a fatty acid or alcohol containing from about 6 to 30, preferably from about 14 to 22 carbon atoms. Examples of triesters include triarachidin, tributyl citrate, tri C₁₂₋₁₃ alkyl citrate, tricaprylin, tricaprylyl citrate, tridecyl behenate, trioctyldodecyl citrate, tridecyl behenate, tridecyl cocoate, tridecyl isononanoate, triisostearyl citrate, and so on.

A variety of other oils may also be used in the composition, including but not limited to animal, vegetable, or mineral oils, which may be synthetic or obtained from naturally occurring sources. Examples of such oils include, but are not limited to, sesame oil, peanut oil, mineral oil, squalene, jojoba oil, and so on.

G. Surfactants

It may be desirable to include one or more surfactants in the composition. If present, such surfactants may be silicone or organic surfactants, and may range from about 0.01-30%, preferably from about 0.05-25%, more preferably from about 0.1-20% by weight of the total composition.

1. Silicone Surfactants

Preferred nonionic silicone surfactants include those having at least one hydrophilic radical and at least one lipophilic radical. These silicone surfactants may be a liquid or solid at room temperature and are water-in-oil or oil-in-water type surfactants that have a Hydrophile/Lipophile Balance (HLB) of about 2 to 18. Preferably the silicone surfactant is a nonionic surfactant having an HLB of about 2 to 12, preferably about 2 to 10, most preferably about 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group portion.

The polymeric silicone surfactant used in the composition may have any of the following general formulas:
MₓQ_{y}, or
MₓT_{y}, or
MDₓD′_{y}D″_{z}M
wherein:
each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof;
T is a trifunctional siloxy unit having the empirical formula R′SiO_{1.5} or RSiO_{1.5} wherein R is methyl and R′ is a C₂₋₂₂ alkyl or phenyl.
Q is a quadrifunctional siloxy unit having the empirical formula SiO₄/2; and
D, D′, D″, x, y, and z are as set forth below, with the proviso that the compound contains at least one hydrophilic radical and at least one lipophilic radical. Preferred is a linear silicone of the formula:
   MDₓD′_{y}D″_{z}M
   wherein M=RRRSiO_{1/2}
D=RR SiO_{2/2}
D′=RR′SiO_{2/2}
D″=R′R′SiO_{2/2}
x, y, and z are each independently 0-1000,
where R is methyl or hydrogen, and R′ is a hydrophilic radical or a lipophilic radical, with the proviso that the compound contains at least one hydrophilic radical and at least one lipophilic radical.
   Most preferred is wherein
M=trimethylsiloxy
D=[Si(CH₃)₂—O]_{2/2}
D′=Si[(CH₃)][(CH₂)ₙCH₃]O_{2/2} where n=0-40,
D″=Si [(CH₃)][(CH₂)ₒ—O—PE)]O_{2/2} where PE is (—C₂H₄O )ₐ(—C₃H₆O)_{b}H, o=0-40, a=1-100 and b=1-100, and

More specifically, suitable silicone surfactants have the formula: 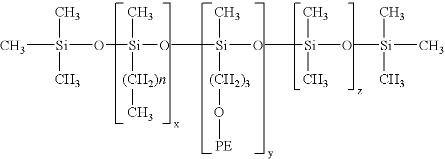
wherein n is 0-40, preferably 12-18, most preferably 14; and
PE is (—C₂H₄O)ₐ(—C₃H₆O)_{b}—H
where x, y, z, a, and b are such that the maximum molecular weight of the polymer is approximately 50,000. An example of such a silicone surfactant is where n=14, having the C.T.F.A. name cetyl dimethicone copolyol. Cetyl dimethicone copolyol may be referred to more specifically by enumerating the number of repeating ethylene oxide and propylene oxide units in the polymer.

Another type of silicone surfactant that may be used in the compositions of the invention are emulsifiers sold by Union Carbide under the Silwet™ trademark, which are referred to by the C.T.F.A. name dimethicone copolyol. One type of dimethicone copolyol may be more specifically referred to as PEG/PPG 18/18 dimethicone, which is dimethicone having 18 PEG (polyethylene glycol) units and 18 PPG (polypropylene glycol) units on the EO (ethylene oxide)/PO (propylene oxide) substituent; or PEG-12 dimethicone, which is a dimethicone having 12 repeating ethylene oxide units on the EO substituted portion of the siloxane backbone.

Also suitable as nonionic silicone surfactants are hydroxy-substituted silicones such as imethiconol, which is defined as a dimethyl silicone substituted with terminal hydroxy groups.

Examples of suitable silicone surfactants are those sold by Dow Coming under the tradename Dow Coming 3225C Formulation Aid, Dow Coming 190 Surfactant, Dow Coming 193 Surfactant, Dow Coming Q2-5200, and the like are also suitable. In addition, surfactants sold under the tradename Silwet by Union Carbide are also suitable. Preferred silicone surfactants for use in the compositions of the invention are dimethicone copolyol or cetyl dimethicone copolyol.

2. Organic Surfactants

The composition may contain one or more organic surfactants either in lieu of, or in combination with, the silicone surfactants mentioned above.

(a). Alkoxylated Alcohols or Ethers

Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Steareth 2-100, formed by the reaction of stearyl alcohol and ethylene oxide where the number of repeating ethylene oxide units ranges from 2 to 100; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, where the number of repeating ethylene oxide units is 1 to 45; laureth 1-100 formed by the reaction of lauryl alcohol and ethylene oxide where the number of repeating ethylene oxide units is 1 to 100; and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol, are also suitable.

(b). Alkoxylated Carboxylic Acids

Also suitable surfactants are alkyoxylated carboxylic acids, which are formed by the reaction of a carboxylic acid with an alkylene oxide or with a polymeric-ether. The resulting products have the general formula: 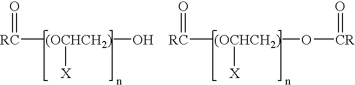
where RCO is the carboxylic ester radical, X is hydrogen or lower C₁₋₄ alkyl, and n is the number of polymerized alkoxy groups. In the case of the diesters, the two RCO-groups do not need to be identical. Preferably, R is a C₆₋₃₀ straight or branched chain, saturated or unsaturated alkyl, and n is from 1-100.

(c). Monomeric or Polymeric Ethers

Suitable surfactants also include monomeric, homopolymeric or block copolymeric ethers. Such ethers are formed by the polymerization of monomeric alkylene oxides, generally ethylene or propylene oxide. Such polymeric ethers have the following general formula: 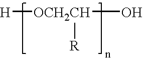
wherein R is H or lower C₁₋₄ alkyl and n is the number of repeating monomer units, and ranges from 1 to 500.

(d). Sorbitan Derivatives

Other suitable nonionic surfactants include derivatives of sorbitan, for example formed by the alkoxylation of sorbitan, or by the reaction of C₁₋₂₅, preferably C₆₋₂₀ carboxylic acids with sorbitol or hexitol anhydrides derived from sorbitol.

For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85.

Examples of sorbitan derivatives include the reaction product of sorbitol or the hexitol anhydrides thereof with fatty acids to form derivative such as sorbitan oleate, sorbitan palmitate, sorbitan sesquiisostearate, sorbitan stearate, sorbitan sesquioleate, and so on.

H. Other Ingredients

The composition may contain a variety of other ingredients that enhance aesthetics, commercial acceptability, or provide other desirable properties. Examples of such ingredients include, but are not limited to preservatives, antioxidants, botanicals, fragrance, and the like.

The compositions of the invention preferably have a refractive index ranging from about 1.0 to 1.5, more preferably from about 1.2 to 1.5, most preferably from about 1.3 to 1.5 at 25° C.

Further, the compositions of the invention preferably have a turbidity reading ranging from 0 to 5 NTU, more preferably from about 0 to 4, most preferably from about 0 to 3 NTU at 25° C.

II. The Method

The invention further comprises a method for improving the clarity of clear gel emulsion antiperspirant compositions having water and oil phases by formulating the compositions such that both the separate oil phase and the water phase prior to emulsification are optically clear. This is achieved by using ingredients in both the water and oil phases that provide a phase mixture that has an index of refraction ranging from about 1.0 to 1.5, preferably from about 1.2 to 1.5, most preferably from about 1.3 to 1.5; and a turbidity reading of from about 0 to 5 NTU, more preferably from about 0 to 4 NTU, most preferably from about 0-3 NTU.

Then when these two clear phases are emulsified together, the resulting antiperspirant composition will also be optically clear and have a refractive index and turbidity reading within the ranges mentioned, and will remain optically clear for a longer period of time.

The preferred oils for use in the oil phase to achieve the improved clarity compositions are those set forth herein under the heading “high refractive index oils”, and are used in the general amounts stated.

The invention will be further described in connection with the following examples, which set forth for the purposes of illustration only.

### EXAMPLE 1

Antiperspirant compositions in the clear gel form were prepared as follows:

| 1 | Ingredient | A | B | C | D | E | F |
|---|---|---|---|---|---|---|---|
| 1 | Cyclopentasiloxane, | 9.90 | 9.90 | 9.00 | 8.70 | 9.00 | 9.00 |
| | PEG/PPG-18/18 | | | | | | |
| | dimethicone | | | | | | |
| 1 | Dimethicone (100 | 3.00 | — | — | — | — | — |
| | centistokes) | | | | | | |
| 1 | Polydecene-2* | — | — | 3.00 | 3.00 | 3.00 | 3.00 |
| 1 | Polydecene-4** | 3.50 | 3.50 | — | — | — | — |
| 2 | Water | 11.60 | 5.60 | 8.00 | 8.00 | 8.00 | 8.00 |
| 2 | Aluminum chlorohydrate | 62.00 | 62.00 | 62.00 | 62.00 | 62.00 | 62.00 |
| | (33% aqueous solution) | | | | | | |
| 2 | Propylene glycol | — | — | — | 16.00 | — | — |
| 2 | Dipropylene glycol | 10.00 | 19.00 | 18.00 | — | 16.00 | 16.00 |
| 2 | Glycerin | — | — | — | 2.00 | 2.00 | 2.00 |
| 2 | Ethylene brassylate | — | — | — | 0.30 | — | — |

*Puresyn-2
**Puresyn-4

The compositions were prepared by separately combining the sequence 1, oil phase ingredients. Separately, the sequence 2, water phase, ingredients were combined. Both phases were optically clear. The two phases were emulsified to form a final liquid mixture that was clear when viewed with the naked eye.

### EXAMPLE 2

Antiperspirant compositions in the clear gel form were prepared as follows:

| | Ingredient | A | B | C | D | E | F |
|---|---|---|---|---|---|---|---|
| 1 | Cyclopentasiloxane, | 9.00 | — | — | — | 9.00 | — |
| | PEG/PPG-18/18 | | | | | | |
| | dimethicone | | | | | | |
| 1 | PEG-12 Dimethicone* | — | — | — | 5.00 | — | — |
| 1 | Cyclopentasiloxane (and) | — | 2.50 | 1.00 | — | — | 2.00 |
| | PEG/PPG-20/15 | | | | | | |
| | Dimethicone** | | | | | | |
| 1 | Cyclopentasiloxane | 3.00 | 6.50 | 9.00 | 5.00 | — | 7.00 |
| 1 | Polydecene-2*** | 3.00 | 6.00 | 5.00 | 5.00 | 3.00 | 3.00 |
| 2 | Water | 5.00 | 5.00 | 5.00 | 5.00 | 8.00 | 8.00 |
| 2 | Aluminum chlorohydrate | 62.00 | 62.00 | 62.00 | 62.00 | 62.00 | 62.00 |
| | (33% aqueous solution) | | | | | | |
| 2 | Dipropylene glycol | 16.00 | 16.00 | 16.00 | 16.00 | 16.00 | 16.00 |
| 2 | Glycerin | 2.00 | 2.00 | 2.00 | 2.00 | 2.00 | 2.00 |

*GE Silicones SF 1450
**GE Silicones SF 1540
***Puresyn 2

The compositions were prepared by combining the sequence 1, oil phase ingredients. The mixture was optically clear. The sequence 2, water phase ingredients were separately combined and formed an optically clear mixture. The two phases were combined and mixed well to form a clear liquid that was clear when viewed with the naked eye.

### EXAMPLE 3

Antiperspirant compositions in the clear gel form were prepared as follows:

| | Ingredient | A | B | C |
|---|---|---|---|---|
| 1 | Cyclopentasiloxane, PEG/PPG- | 9.00 | 9.00 | 9.00 |
| | 18/18 dimethicone | | | |
| 1 | Polydecene-2* | 3.00 | 6.00 | 3.00 |
| 1 | Fragrance | 0.70 | — | — |
| 1 | Ethylene brassylate | 0.001 | 0.50 | 0.50 |
| 2 | Water | QS | QS | QS |
| 2 | Aluminum chlorohydrate | 62.00 | 62.00 | 62.00 |
| | (33% aqueous solution) | | | |
| 2 | Dipropylene glycol | 16.60 | 16.60 | 16.60 |
| 2 | Glycerin | 2.00 | 2.00 | 2.00 |

*Puresyn 2

The compositions were prepared by combining the sequence 1, oil phase ingredients. The mixture was optically clear. The sequence 2, water phase, ingredients were separately combined and formed an optically clear mixture. The two phases were combined and mixed well to form a clear liquid.

### EXAMPLE 4

Antiperspirant compositions in the clear gel form were prepared as follows:

| | Ingredient | A | B |
|---|---|---|---|
| 1 | Cyclopentasiloxane, PEG/PPG- | 9.00 | 9.00 |
| | 18/18 dimethicone | | |
| 1 | Hydrogenated didecene | — | 3.00 |
| 1 | Hydrogenated polydecene | 3.00 | — |
| 1 | Ethylene brassylate | 0.50 | 0.50 |
| 2 | Water | QS | QS |
| 2 | Aluminum chlorohydrate | 62.00 | 62.00 |
| | (33% aqueous solution) | | |
| 2 | Dipropylene glycol | 16.60 | 16.60 |
| 2 | Glycerin | 2.00 | 2.00 |

The compositions were prepared by combining the sequence 1, oil phase ingredients. The mixture was optically clear. The sequence 2, water phase, ingredients were separately combined and formed an optically clear mixture. The two phases were combined and mixed well to form a clear liquid.

### EXAMPLE 5

A deodorant composition was made as follows:

| | Ingredient | A |
|---|---|---|
| 1 | Cyclopentasiloxane, PEG/PPG- | 9.00 |
| | 18/18 dimethicone | |
| 1 | Hydrogenated didecene | 3.00 |
| 1 | Triclosan | 0.20 |
| 1 | Fragrance | 1.00 |
| 2 | Water | QS |
| 2 | Dipropylene glycol | 16.60 |
| 2 | Glycerin | 2.00 |

The compositions were prepared by combining the sequence 1, oil phase ingredients. The mixture was optically clear. The sequence 2, water phase, ingredients were separately combined and formed an optically clear mixture. The two phases were combined and mixed well to form a clear liquid.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A clear gel emulsion antiperspirant or deodorant composition comprising at least one antiperspirant or deodorant active, a water phase, and an oil phase containing at least one high refractive index oil, wherein the composition is prepared from an oil phase and a water phase that, prior to emulsification, are both optically clear.

2. The composition of claim 1, which is a deodorant.

3. The composition of claim 2, which is in the water in oil emulsion form.

4. The composition of claim 3 wherein the high refractive index oil is one or more polymeric alpha olefins.

5. The composition of claim 4 wherein the water phase comprises water and at least one water soluble solvent.

6. The composition of claim 1 which is an antiperspirant comprising at least one antiperspirant active salt.

7. The composition of claim 6, which is a water in oil emulsion.

8. The composition of claim 7 wherein the water phase comprises from about 0.1-95% by weight of the total composition.

9. The composition of claim 7 wherein the high refractive index oil in the oil phase is one or more polymeric alpha olefins.

10. The composition of claim 8 wherein the at least one polymeric alpha olefin comprises from about 0.1-95% by weight of the total composition.

11. The composition of claim 7 wherein the water phase comprises water and at least one water soluble solvent.

12. The composition of claim 11 wherein the water soluble solvent comprises a mono-, di-, or polyhydric alcohol or a polymeric C₁₋₆ alkylene glycol.

13. The composition of claim 11 wherein the water soluble solvent comprises a polymeric C₁₋₆ alkylene glycol, or a polyhydric alcohol.

14. The composition of claim 13 wherein the polymeric C₁₋₆ alkylene glycol is dipropylene glycol and the polyhydric alcohol is glycerin.

15. The composition of claim 6 wherein the antiperspirant actives are one or more astringent antiperspirant salts solubilized or dispersed in the aqueous phase.

16. The composition of claim 15 wherein the astringent antiperspirant salts are solubilized in the aqueous phase.

17. The composition of claim 6 wherein the salts are chloride salts of aluminum, zirconium, or mixtures thereof.

18. The composition of claim 17 wherein the astringent antiperspirant salt comprises aluminum chlorohydrate.

19. The composition of claim 1 wherein the oil phase comprises from about 0.01-25% by weight of the total composition of at least one high refractive index oil comprising one or more polymeric alpha olefins.

20. The composition of claim 19 wherein the polymeric alpha olefins are polymers of C₁₋₁₂ alpha olefin monomers.

21. The composition of claim 20 wherein the C₁₋₁₂ alpha olefin monomers are butene, pentene, hexene, octene, decene, dodecene, or tetradecene.

22. The composition of claim 21 wherein the number of repeating alpha olefin monomers ranges from 2 to 500.

23. The composition of claim 19 wherein the polymeric alpha olefin has a refractive index ranging from about 1.1 to 1.5.

24. The composition of claim 1 having a nephelometric turbidity reading ranging from 0 to 5 TU.

25. The cornposition of claim 1 having a refractive index ranging from about 1.0 to 1.5.

26. A clear gel emulsion antiperspirant or deodorant composition comprising at least one antiperspirant or deodorant active, a water phase, and an oil phase, wherein the oil phase contains at least one polymeric alpha olefin.

27. The composition of claim 26 wherein the polymeric alpha olefin comprises polybutene, polydecene, or mixtures thereof.

28. The composition of claim 26 wherein the water phase comprises at least one water soluble solvent.

29. The composition of claim 26, which is an antiperspirant.

30. The composition of claim 29, comprising at least one halide or halohydrate salt of aluminum.

31. A method improving the clarity of a clear gel emulsion antiperspirant or deodorant composition comprising separately preparing an optically clear water phase and an optically clear oil phase containing at least one high refractive index oil, and emulsifying the phases to provide an optically clear final composition.

32. The method of claim 31 wherein the oil phase comprises at least one high refractive index oil in an amount sufficient to provide an optically clear oil phase prior to emulsifying the phases.

33. The method of claim 32 wherein the high refractive index oil comprises a polymeric alpha olefin.

34. The method of claim 33 wherein the polymeric alpha olefin is polydecene, polybutene, or mixtures thereof.

35. The method of claim 30 wherein the composition is an antiperspirant.
